(19) United States
(12) Patent Application Publication
(10) Pub. No.: US 2011/0125105 A1
(43) Pub. Date: May 26, 2011

(54) CONDUIT PROTECTOR

(75) Inventors: James L. Pokorney, Northfield, MN (US); Kemal Schankereli, Stillwater, MN (US)

(73) Assignee: CARDIOUS, INC., St. Paul, MN (US)

(21) Appl. No.: 12/674,827

(22) PCT Filed: Aug. 22, 2008
(86) PCT No.: PCT/US08/74044

§ 371 Date: Feb. 23, 2010

Related U.S. Application Data
(60) Provisional application No. 60/965,960, filed on Aug. 23, 2007.

(51) Int. Cl. A61M 39/00; B32B 1/08

(52) U.S. Cl. 604/263; 428/34.1

(57) ABSTRACT

A conduit protector for temporarily minimizing the surface friction on an outside surface of an implant includes a curvilinear sheet formed into a generally cylindrical shape having a first axial edge and a second axial edge, a connecting member coupling the first axial edge of the curvilinear sheet to the second axial edge of the curvilinear sheet, and a release member structured for separating the first axial edge of the curvilinear sheet from the second axial edge of the curvilinear sheet. An inside surface of the curvilinear sheet may have an inner diameter approximately equal to an outer diameter of the outside surface of the implant.

### CROSS-REFERENCE TO RELATED APPLICATIONS

This application claims the benefit of Provisional Application Ser. No. 60/965,960, filed on Aug. 23, 2007, the entirety of which is hereby incorporated by reference.

### FIELD OF THE INVENTION

This invention relates generally to a conduit protector that can be used with a conduit implant. More particularly, the invention relates to a disposable conduit protector that allows for low friction, atraumatic insertion of a conduit or other implant into the wall of a beating heart or other body organ.

### BACKGROUND OF THE INVENTION

A reduction in the cardiac output of the heart (i.e., the reduced ability of the heart to output oxygenated blood from the left side of the heart) can result from various abnormalities and diseases. In most cases, this reduction in output is due to aortic valve disease. The major type of aortic heart valve disease is valve stenosis, which involves the narrowing of the aortic outflow tract. Typically, stenosis involves the buildup of calcified material on the valve leaflets, causing them to thicken and impairing their ability to fully open to permit adequate forward blood flow.

Stenosis of the aortic valve may obstruct flow leaving the ventricle. This obstruction of the outflow tract can ultimately lead to hypertrophy of the left ventricle, meaning the size of the ventricular chamber becomes enlarged. This condition leads to diastolic dysfunction of the left ventricle, which is an impaired ability of the left ventricle to adequately fill with blood. Historically, such diastolic dysfunction accounts for about 20% to 40% of heart failures.

Open heart surgical treatment is available to relieve left ventricular outflow tract obstruction due to stenosis. In most cases, the native aortic valve is surgically removed and replaced with a prosthetic or man-made valve. Valve replacement surgery has been performed for over 40 years and is considered the most effective therapy for outflow tract obstruction even though the technique has numerous drawbacks.

One drawback of the conventional aortic valve replacement procedure is that it requires the patient to be placed on a heart-lung machine wherein the heart and lungs are stopped. Open-heart surgery on a still heart involves the use of cardiopulmonary bypass, aortic cross-clamping, and cardioplegic arrest. The risks and complications associated with this highly invasive procedure are well known. The most serious risks of cardiopulmonary bypass and aortic cross-clamping include an increase in the likelihood of bleeding and stroke. A stroke, which is an occlusion of an artery in the brain, can be caused by particles or emboli generated during a heart valve procedure. Emboli can be generated as calcific particles resulting from the necessary manipulation of a calcified aorta or valve. Alternatively, emboli can be generated in the form of blood clots caused by the interaction of the blood with the foreign surfaces of the heart-lung machine.

Another drawback of the conventional aortic valve replacement procedure is that patients who undergo such a procedure using cardiopulmonary bypass often require extended hospital stays and experience lengthy recoveries. As a result, conventional aortic valve replacement procedures can be very costly.

Yet another drawback of conventional aortic valve replacement procedures resides in the fact that the patient's heart must be stopped. Thus, while these procedures may produce beneficial results, numerous people who might benefit from such a procedure are unable or unwilling to undergo the trauma and risks of a conventional stopped heart procedure.

Tools and techniques for the interposition of an extracardiac conduit between the left ventricle and the aorta have been evolving over the last century. For example, a less invasive means to implant a prosthetic heart valve housing is described in U.S. Patent Application Publication Nos. 2005/0149093, 2007/0055357, and 2008/0009895, which are hereby incorporated by reference in their entireties into the present application. Generally speaking, these publications describe an innovative implant, implantation tools, and corresponding implantation method that substantially reduce the potential for excessive blood loss or the generation of stroke causing emboli. Furthermore, the publications describe an implant, tools, and implantation method that reduce the possibility of inflicting damage to the heart, maximize blood flow through the implant, and protect the implant from kinking or crushing blows.

To those knowledgeable in the art of insertion of a conduit or other implantable device intended for permanent implantation into a heart wall or any other body organ, there are two primary design goals. First, the implant should preferably slide into place easily and without trauma to the adjacent tissue during the insertion process. Second, once in place the implant should have a surface conducive to permanent in-growth and attachment of the surrounding tissue. Unfortunately, a surface that is optimal for insertion is typically not the type of surface that is optimal for long term compatibility with human tissue. For instance, an insertion surface may preferably have a generally low coefficient of friction, while an implant surface for permanent attachment may preferably have a high coefficient of friction.

Examples of aortic valve bypass conduits exhibiting both design goals may be found. In the 1970's, Dr. Cooley and associates at Texas Heart Hospital employed a conduit connector designed for easy insertion into the wall of the heart. This connector was composed of pyrolytic carbon, and thus, could be easily inserted into the heart wall due to pyrolytic carbon's smooth, highly polished surface. However, as reported by Dr. Cooley, the smooth connector did not heal sufficiently into the heart wall and became dislodged months after surgery, causing immediate death of the patient. In the 1980's, Dr. Pierce and associates in Indiana employed a conduit covered in polyester. This connector, due to the multi-filament textile nature of its surface, exhibited excellent attachment and healing properties to the adjacent tissue, but was difficult to insert because of the high coefficient of friction inherent in textiles. To accommodate this difficulty in implant insertion, the hole created in the heart wall must be approximately the size of the implant. However, a hole that is even slightly oversized creates the possibility of a blood leak around the connector, which may in turn cause the formation of an aneurysm-like pocket of blood.

In summary, previous implantable conduit designs have neither contemplated nor specifically addressed how to provide an implant device that may be easily inserted into the target region (low coefficient of friction) but that also provides a safe and stable long term implant surface (high coefficient of friction). Thus, although present day devices (such as those referenced above) provide key enabling technologies that allow mainstream use of a valve bypass graft procedure, an improved method of inserting a graft into a beating heart is desirable in order to accommodate both design goals and provide a safer and more effective procedure. In particular, what is needed is a device that provides a temporary low friction surface on the outside surface of an implantable conduit that may be removed after insertion of the conduit to reveal an exterior conduit surface having a generally higher coefficient of friction and other characteristics designed specifically for long term implantation.

### BRIEF SUMMARY OF THE INVENTION

The present invention solves the foregoing problems by providing a conduit protector for temporarily minimizing the surface friction on an outside surface of an implant that includes a curvilinear sheet formed into a generally cylindrical shape having a first axial edge and a second axial edge, a connecting member coupling the first axial edge of the curvilinear sheet to the second axial edge of the curvilinear sheet, and a release member structured for separating the first axial edge of the curvilinear sheet from the second axial edge of the curvilinear sheet. An inside surface of the curvilinear sheet may have an inner diameter approximately equal to an outer diameter of the outside surface of the implant.

One object of the present invention is to facilitate a low friction, atraumatic insertion of a conduit or graft implant through the wall of a beating heart or other body organ. Specifically, the present invention provides many advantages, including but not limited to: providing a temporary low friction surface on the outside surface of an implant to facilitate insertion of the implant into a body organ without excessive trauma to the body organ tissue in contact with the implant; simplifying the insertion of a textile covered implant into a body organ by temporarily reducing the coefficient of friction between the implant and the body organ; allowing for the use of an implant having an exterior surface designed specifically for long term implantation characteristics without requiring the surface to have insertion characteristics such as a low coefficient of friction; allowing a generally curvilinear sheet of low friction material covering the outside of a high friction implant to be easily removed from the implant after it has been inserted into a body organ; and simplifying the removal of a conduit protector having a low friction surface by using a release member to convert the protector's configuration from a generally cylindrical shaped member to a curvilinear sheet prior to removing the protector from the implant.

The above mentioned objects and advantages of this invention will become apparent from the following description taken in connection with the accompanying drawings, which set forth by way of illustration and example, exemplary embodiments of the present invention.

### BRIEF DESCRIPTION OF THE DRAWINGS

FIG. 1 is a perspective view illustrating one exemplary embodiment of a conduit protector in accordance with the present invention.

FIG. 2 is a diagram illustrating exemplary components that may be used to construct the conduit protector of FIG. 1.

FIG. 3 is an exploded perspective view illustrating the conduit protector of FIG. 1 and an implant graft structured to receive the conduit protector.

FIG. 4 is a perspective view illustrating the conduit protector of FIG. 1 positioned on an end of the implant graft.

FIG. 5 is a diagram illustrating a first exemplary step of using a conduit protector in accordance with the present invention.

FIG. 6 is a diagram illustrating a second exemplary step of using a conduit protector in accordance with the present invention.

FIG. 7 is a diagram illustrating a third exemplary step of using a conduit protector in accordance with the present invention.

FIG. 8 is a diagram illustrating a fourth exemplary step of using a conduit protector in accordance with the present invention.

FIGS. 9A-9L are diagrams illustrating the conduit protector of FIG. 1 in accordance with the present invention in use with an implant graft and vessel cutter.

FIGS. 10A-10B are diagrams illustrating the components used to construct a second exemplary embodiment of a conduit protector in accordance with the present invention.

FIG. 11 is an exploded perspective view illustrating the conduit protector of FIGS. 10A-10B and an implant graft structured to receive the conduit protector.

FIG. 12 is a perspective view illustrating the conduit protector of FIGS. 10A-10B positioned on an end of the implant graft.

FIG. 13 is a diagram illustrating the conduit protector of FIGS. 10A-10B being removed from the implant graft.

FIG. 14 is a perspective view of the conduit protector of FIGS. 10A-10B separated from the implant graft.

FIG. 15 is a perspective view of a third alternative embodiment of a conduit protector in accordance with the present invention.

FIG. 16 is a cross-sectional view of a distal region of the conduit protector shown in FIG. 15 and an adaptor portion of an implant graft.

FIG. 17 is a perspective view of a fourth alternative embodiment of a conduit protector in accordance with the present invention.

FIG. 18 is a cross-sectional view of a distal region of the conduit protector shown in FIG. 17.

### DETAILED DESCRIPTION OF THE INVENTION

Generally speaking, the present invention includes a conduit protector that provides a temporary, low friction surface on an outer surface of a generally cylindrical shaped implant to enable simple insertion of the implant into the heart or other organ with minimal trauma. Once inserted, the low coefficient of friction protector may be removed, thereby allowing a higher coefficient of friction surface residing on the implant to be in contact with the heart. Thus, the conduit protector may be used during insertion to provide a “slippery” surface to guide the implant into place with minimal trauma, but may thereafter be removed to expose an outer surface of the implant that may be designed to provide a superior biocompatible interface with the heart tissue for long term implantation.

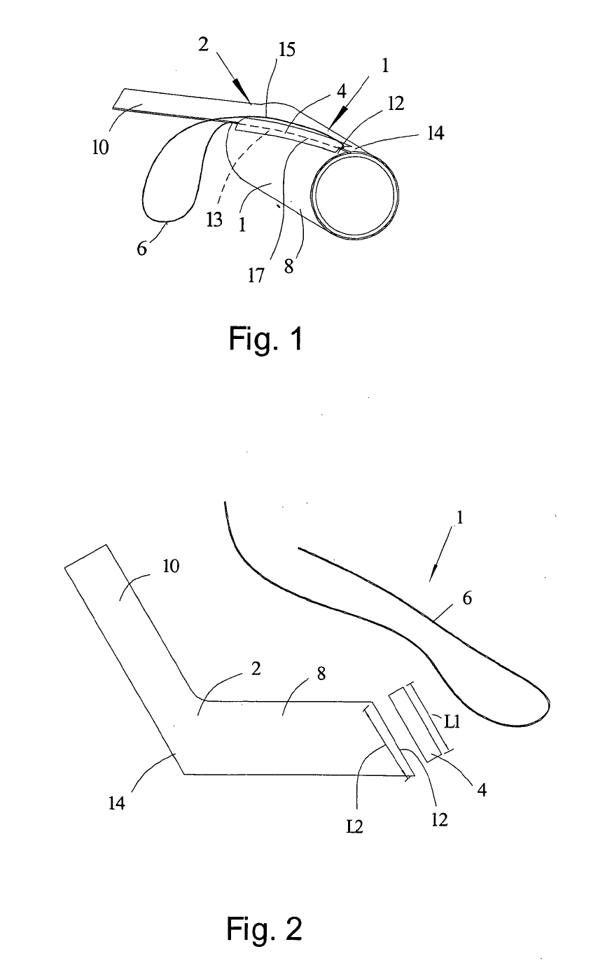

FIG. 1 is a perspective view of one exemplary embodiment of conduit protector 1 in accordance with the present invention, while FIG. 2 is a top view illustrating exemplary components that may be used to construct conduit protector 1. As illustrated in FIG. 1, conduit protector 1 generally includes sheet 2, connecting member 4, and release member 6. In particular, sheet 2 includes body portion 8, handle 10, leading edge 12 at a first end of body 8, and trailing edge 14 at a second end of body 8. Body 8 of sheet 2 is structured to form a generally cylindrical structure with handle portion 10 and release member 6 extending from an end thereof.

As illustrated in FIG. 2, prior to being rolled into the generally cylindrical structure shown in FIG. 1, body 8 and handle 10 of sheet 2 form a generally “L” shaped member with a leading 12 at the first end of body 8 and trailing edge 14 at the second end of body 8. In one exemplary embodiment, the length L1 of connecting member 4 is equal to or less than the length L2 of leading edge 12 of body 8, although such a relationship is not a necessary component of the present invention. Alternatively, instead of a single connecting member 4, multiple smaller connecting members 4 may instead be utilized.

In order to construct conduit protector 1 as illustrated in FIG. 1 with the various components illustrated in FIG. 2, sheet 2 may initially be formed into a cylinder, with leading edge 12 substantially abutting or slightly overlapping trailing edge 14. In this position, a first portion 13 of release member 6 may be aligned with leading and trailing edges 12 and 14, respectively, while connecting member 4 may be applied over that portion of release member 6 such that the connecting member is in contact with both leading edge 12 and trailing edge 14. Securing leading edge 12 to trailing edge 14 with connecting member 4 creates the generally cylindrical structure illustrated in FIG. 1 with the portion of release member 6 disposed beneath connecting member 4. As further illustrated in FIG. 1, a second portion 15 of release member 6 may be positioned adjacent to and extend longitudinally along outer surface 17 of connecting member 4. As will be discussed in further detail to follow, release member 6 may be used to cut or tear through connecting member 4 to separate leading edge 12 from trailing edge 14 during removal of conduit protector 1 from an implant.

Sheet 2 may be formed from any suitable material including, but not limited to, polycarbonate, nylon, mylar, or numerous other low coefficient of friction polymers. In one exemplary embodiment, sheet 2 has a thickness generally in a range between about 0.001 inches and 0.020 inches, although numerous other thicknesses are also contemplated. Furthermore, the suitable thickness of sheet 2 may depend upon the particular application of conduit protector 1.

Connecting member 4 may comprise numerous connecting means in various embodiments. For example, in one exemplary embodiment, connecting member 4 may comprise a strip of material having adhesive applied to one side, such as a strip of adhesive tape. Particularly, the connecting member may be formed from a low friction polymer such as mylar or polycarbonate film with an adhesive applied to one side. In another exemplary embodiment, connecting member 4 may comprise an adhesive applied directly to leading edge 12 and trailing edge 14 that is structured to temporarily adhere the edges together. In yet another exemplary embodiment, the connecting function could be realized by using a curvilinear tube having a perforated section as a connecting member that could be separated using a release member. Thus, as will be appreciated by those skilled in the art, any suitable connecting means that may temporarily connect leading edge 12 to trailing edge 14 is contemplated and within the intended scope of the present invention.

Release member 6 may also incorporate numerous different structures in various embodiments. In one exemplary embodiment, release member 6 may comprise a release member formed from any suitable thread or filament material. Examples of suitable materials include, but are not limited to, polyester, polyethylene, poly-paraphenylene terephthalamide (Kevlar®), stainless steel, or nitinol. Furthermore, release members in accordance with the present invention may be manufactured as a continuous loop member, or alternatively may be formed from one or more strands of material that are tied or otherwise coupled together to form a loop-shaped member, such as with an adhesive or by heat welding. As will be appreciated by those skilled in the art, any suitable release means that may be used for separating leading edge 12 from trailing edge 14 is contemplated and within the intended scope of the present invention.

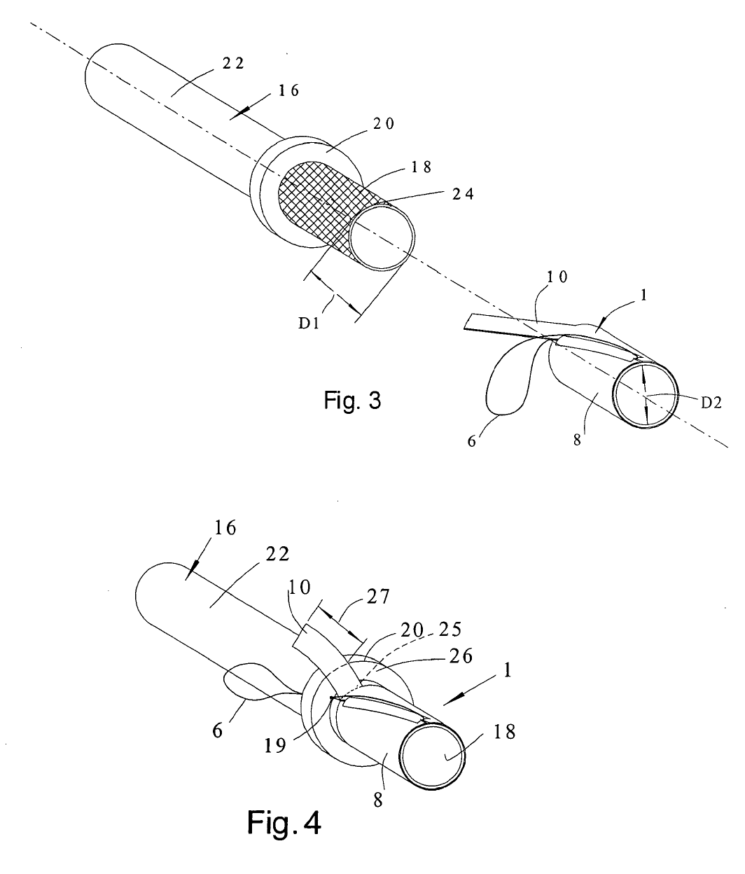

FIG. 3 is an exploded perspective view illustrating conduit protector 1 positioned adjacent implant graft 16, which is one exemplary embodiment of an implant usable with the conduit protector in accordance with the present invention. However, those skilled in the art will appreciate that conduit protectors in accordance with the present invention may be used in conjunction with numerous other types of implants and devices without departing from the intended scope of the present invention. Thus, implant graft 16 will be discussed merely for purposes of example and not for limitation.

As illustrated in FIG. 3, implant graft 16 generally includes adaptor 18, cuff 20, and graft 22. Adaptor 18 may be covered with a high friction material 24, as illustrated by the cross-hatched area in FIG. 3. In one exemplary embodiment, high friction material 24 may comprise a polyester knit textile. However, numerous other materials are contemplated that are designed to provide a superior biocompatible interface with the heart tissue for long term implantation including, but not limited to, woven or non-woven substrates composed of polyester or polytetrafluoroethylene (Teflon) with or without hydrogel or carbon coatings.

In one exemplary embodiment, the high friction material portion 24 may be formed as an integral portion of adaptor 18. Alternatively, high friction material portion 24 may be formed as a generally cylindrical member that is attachable to the generally cylindrical adaptor portion 18 of implant graft 16. Preferably, the outer diameter D1 of high friction material portion 24 of adaptor 18 may be sized to fit closely to the inner diameter D2 of conduit protector 1 to minimize the sliding of conduit protector 1 relative to adaptor 18 upon joining of the two elements.

FIG. 4 is a perspective view illustrating conduit protector 1 positioned on adaptor 18 of implant graft 16. When assembled as illustrated in FIG. 4, a portion of release member 6 may be inserted through cuff 20. In particular, release member 6 may be inserted through an aperture 19 in cuff 20 such that a proximal portion of release member 6 extends toward graft portion 22 of implant graft 16, while a distal portion remains on an opposing side of cuff 20 and is secured by connecting member 4. Alternatively, instead of extending through an aperture 19 in cuff 20, the proximal portion of release member 6 may be positioned adjacent front face 26 of cuff 20 and extended over cuff 20 toward graft portion 22 of implant graft 16.

As further illustrated in FIG. 4, handle 10 of the conduit protector 1 may be folded along a line 25 approximately at the intersection with body 8 such that handle 10 is generally coplanar with front face 26 of cuff 20. Additionally, the length of handle 10 may preferably be designed such that an upper portion 27 of handle 10 extends past front face 26 of cuff 20 to enable a surgeon to grasp handle 10 during removal of conduit protector 1 as will be discussed in further detail to follow.

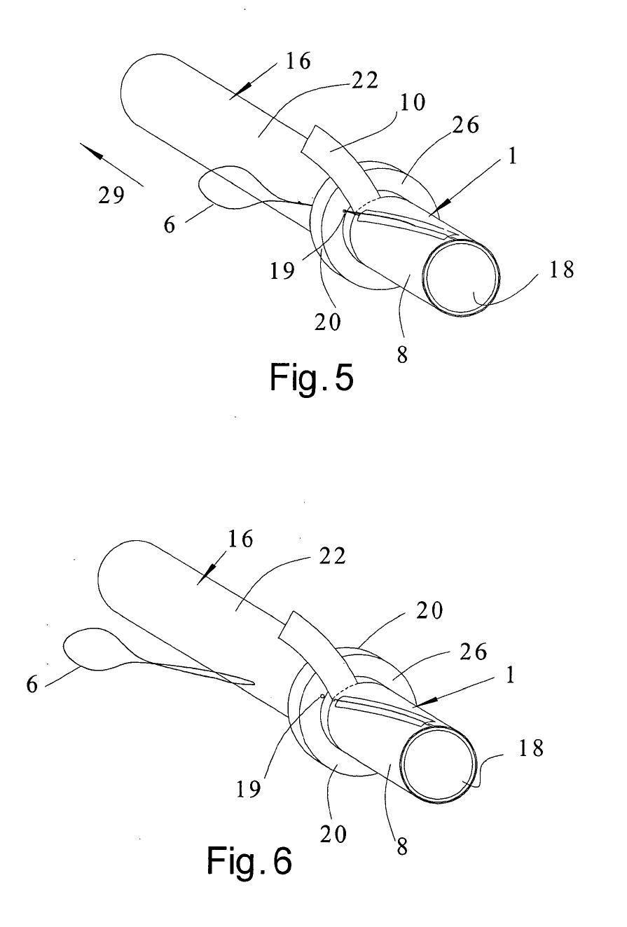

FIGS. 5-8 are diagrams illustrating one exemplary method of using a conduit protector in accordance with the present invention. In particular, FIG. 5 is a diagram illustrating a first exemplary step of using a conduit protector in accordance with the present invention, wherein conduit protector 1 is positioned over adaptor 18 of implant graft 16 as previously discussed in reference to FIG. 4. In this position, conduit protector 1 provides a low friction surface that facilitates insertion of implant graft 16 into a body cavity. As will be described in further detail to follow, after a successful insertion of adaptor 18, conduit protector 1 may be removed to reveal the biocompatible, high friction surface 24 of adaptor 18. Accordingly, once adaptor 18 has been inserted into a target region, the surgeon may begin to pull release member 6 through cuff 20 in the direction generally indicated by arrow 29. The surgeon continues to pull release member 6 until it is completely detached from conduit protector 1, as illustrated in FIG. 6.

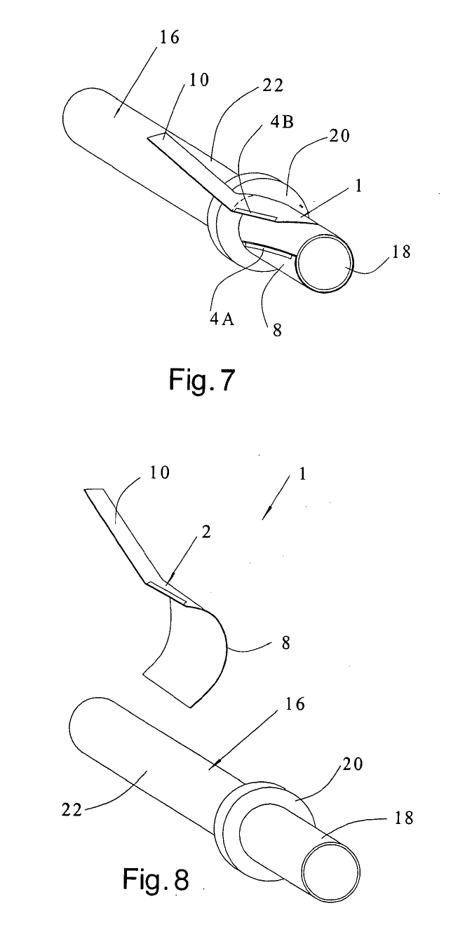

As release member 6 is pulled in the direction indicated by arrow 29, the release member may tear or otherwise cut through connecting member 4. Thus, once release member 6 has been completely detached from conduit protector 1, connecting member 4 is separated into a first connecting member portion 4A and a second connecting member portion 4B as illustrated in FIG. 7. As a result, conduit protector 1 is converted from a generally cylindrical member to a generally curvilinear sheet, which may then be pulled free of implant graft 16 as illustrated in FIG. 8.

As will be appreciated by those skilled in the art, with conduit protector 1 positioned over adaptor 18 of implant graft 16, the implant graft 16 may be implanted into the wall of a heart using, for example a vessel cutting tool. One exemplary embodiment of a vessel cutting tool is shown and described in U.S. Patent Application Publication No. 2008/0009895, which once again is incorporated by reference herein in its entirety. A cutting method using a cutting tool with a shroud on its distal end similar to that disclosed in the '895 publication is hereinafter described in the present application.

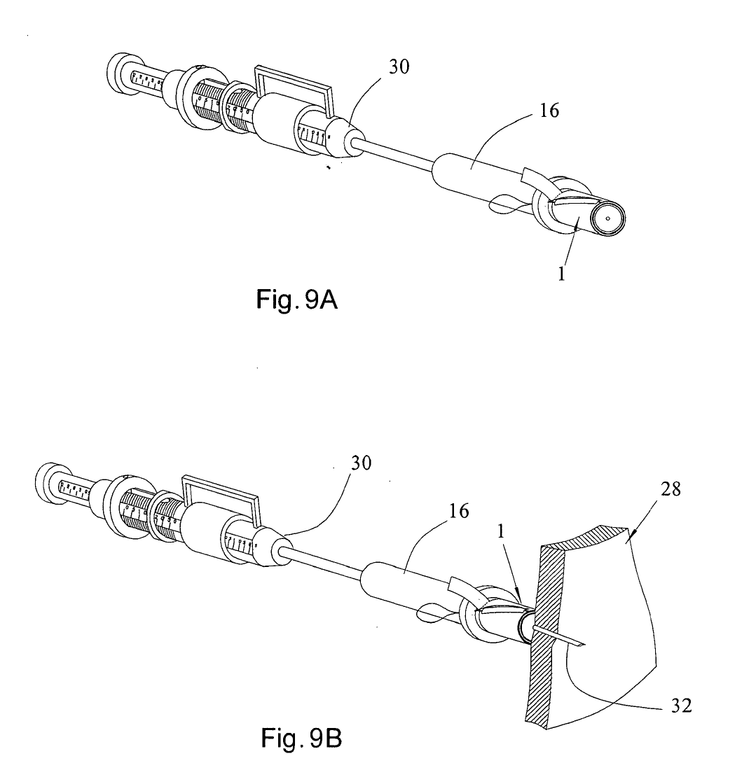

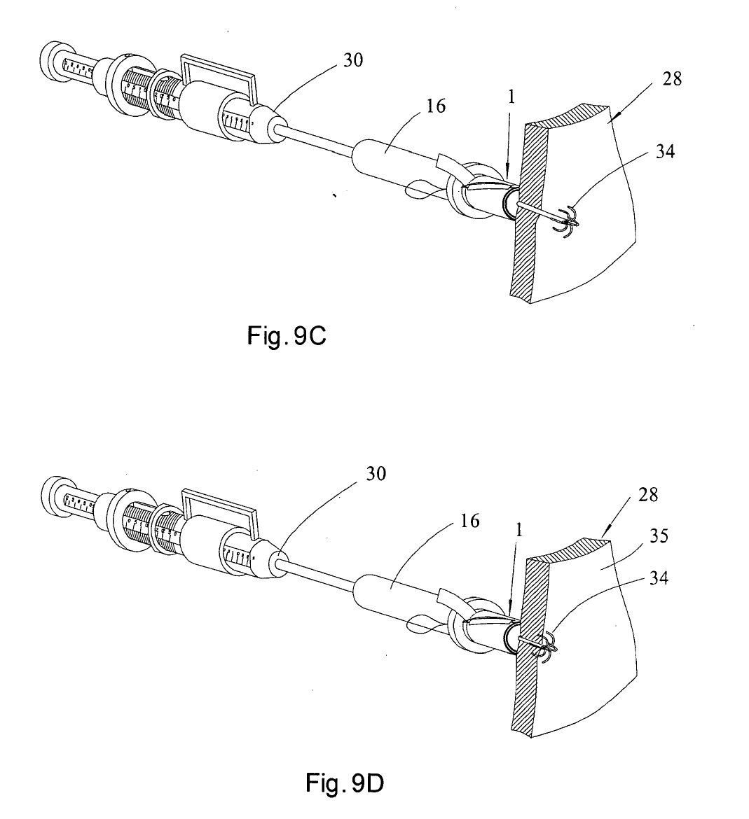

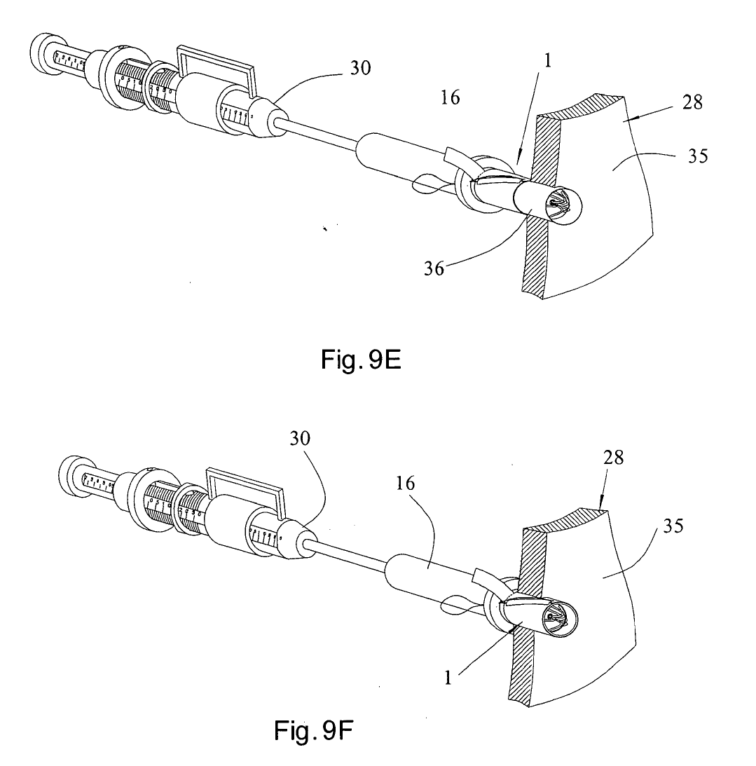

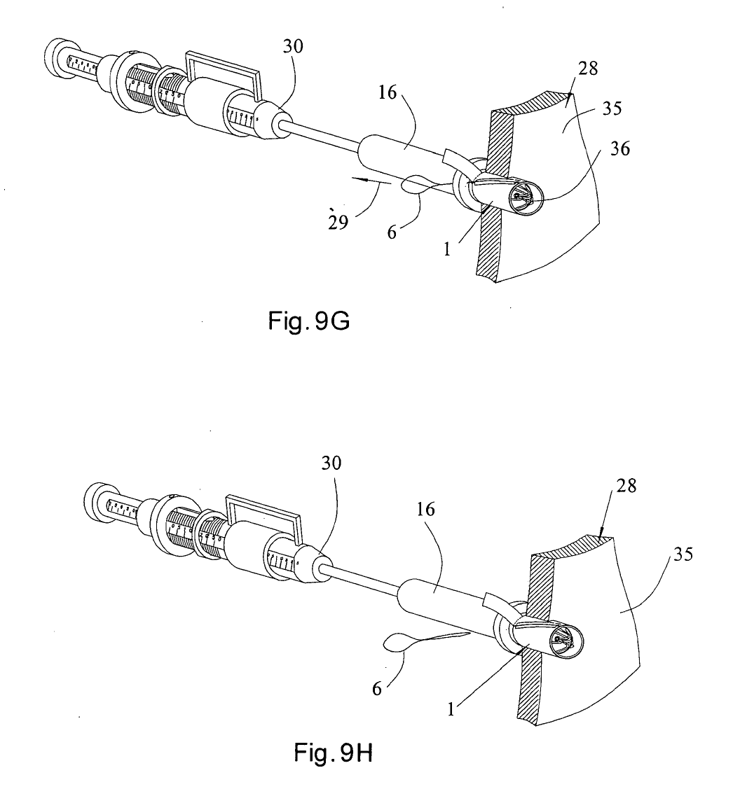

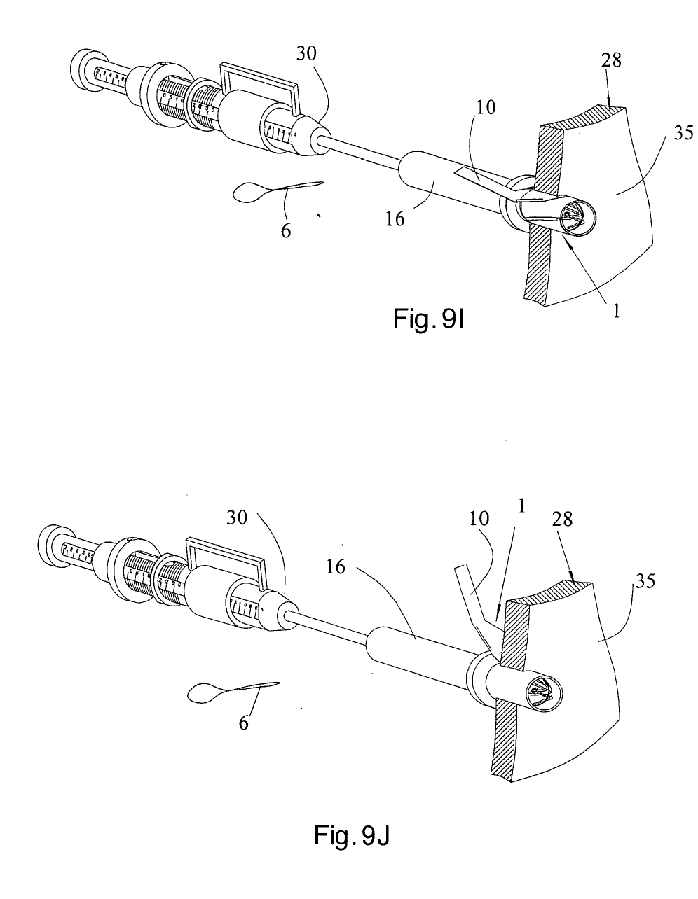

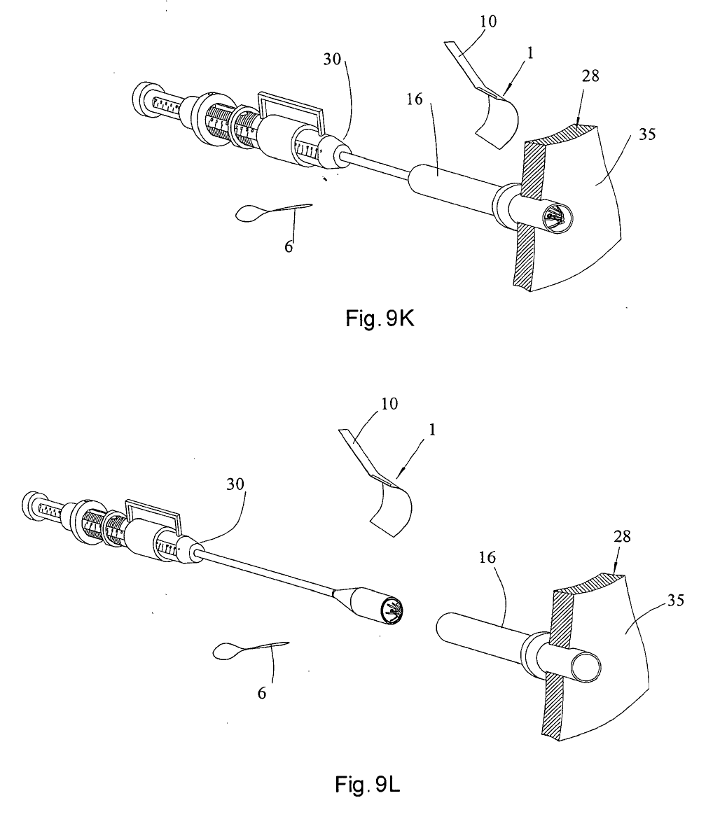

FIGS. 9A-9L are diagrams illustrating one exemplary method of using a conduit protector in accordance with the present invention to facilitate placement of an implant graft into a heart wall. In particular, FIG. 9A illustrates a first step of the method wherein conduit protector 1 is loaded over adaptor 18 of implant graft 16, which in turn is loaded over a distal end of vessel cutting tool 30.

Next, as illustrated in FIG. 9B, the distal end of vessel cutting tool 30 may be placed against heart wall 28. Once positioned against heart wall 28, a cannula 32 of vessel cutting tool 30 may be inserted through heart wall 28. Then, as illustrated in FIG. 9C, a spring loaded anchor 34 of vessel cutting tool 30 may be advanced out of a distal end of cannula 32. Cannula 32 may then be retracted as illustrated in FIG. 9D, thereby allowing the spring loaded anchor 34 to retract against an inside surface 35 of heat wall 28. Once anchor 34 has been retracted, cutting drum 36 of vessel cutting tool 30 may be advanced distally through heart wall 28 as illustrated in FIG. 9E such that a distal end of cutting drum 36 extends past the inside surface 35 of heart wall 28. With cutting drum 36 advanced through heart wall 28, conduit protector 1 and implant graft 16 may be advanced through heart wall 28 as shown in FIG. 9F. In one exemplary process of advancing conduit protector 1 and implant graft 16, conduit protector 1 is first abutted against cuff 20. Then, the surgeon may simultaneously push conduit protector 1 and underlying implant graft 16 through the incision made by cutting drum 36 until the leading end of implant graft 16 is completely advanced through heart wall 28. During the device advancement, the heart wall is in contact with conduit protector 1 instead of implant graft 16. Thus, as will be appreciated by those skilled in the art based upon the present disclosure, this advancement of implant graft 16 is facilitated by the low friction outer surface of conduit protector 1. This low friction surface is particularly important if the diameter of the hole cut by cutting drum 36 is less than the outside diameter of implant graft 16, as is the case in the illustrated embodiment.

Once implant graft 16 has been inserted into heart wall 28, conduit protector 1 may be removed as previously described in reference to FIGS. 5-8. The first step in this removal process is to transform the generally cylinder shape of conduit protector 1 into a curvilinear sheet. As illustrated in FIG. 9G, this may be accomplished in the present embodiment of the invention by pulling back on release member 6 in the direction indicated by arrow 29 until release member 6 is completely detached from conduit protector 1, as illustrated in FIG. 9H.

With release member 6 detached from conduit protector 1, and connecting member 4 being cut so as to separate leading edge 12 from trailing edge 14 of body 8, conduit protector 1 may thereafter be removed by pulling handle 10 as illustrated in FIGS. 9I and 9J. In particular, as the surgeon pulls handle 10 away from the heart, the now curvilinear sheet conduit protector 1 may rotate around implant graft 16 while it slides axially away from heart wall 28. The trailing edge 14 of conduit protector 1 may be removed first, followed by leading edge 12. It should be noted that the curvilinear sheet shape of conduit protector 1 may be pulled out from the space created between heart wall 28 and adaptor 18 of implant graft 16 while maintaining cuff 20 substantially in contact with or in close proximity to heart wall 28. This may be accomplished due to the sheet-like nature of conduit protector 1 as well as the flexibility of the material used to construct the conduit protector. As illustrated in FIG. 9K, continuing to pull handle 10 allows conduit protector 1 to be completely removed from contact with implant graft 16.

Finally, as illustrated in FIG. 9L, anchor 34 and cutting drum 36 may be retracted from heart wall 28. Then, cutting tool 30 may be removed from implant graft 16, leaving implant graft 16 in place adjacent heart wall 28. An optional clamp (not shown) may thereafter be coupled to graft portion 22 of implant graft 16 in order to impede blood flow from the heart.

Based upon the method depicted in FIGS. 9A-9L, it will be apparent to those skilled in the art how conduit protector 1 may be employed to allow quick and easy insertion of a generally high friction textile covered adaptor into a heart wall with minimal trauma to the surrounding wall. Furthermore, it will be apparent to those skilled in the art that removal of conduit protector 1 may be easily accomplished by transforming an otherwise generally cylindrical member wrapped around adaptor 18 into a generally curvilinear sheet of material.

Those skilled in the art will appreciate that in alternative embodiments, the generally cylindrical shaped conduit protector 1 illustrated in FIG. 1 may be transformed into two or more curvilinear sheets. In particular, this alternative embodiment may be created by forming a conduit protector with two or more sheets of, for example, a thin polymer material. Then, two or more sets of connecting members 4 and release members 6 may be formed around the periphery of the conduit protector to create a temporary cylindrical shape. Similar to the embodiment previously described using a single sheet of material, removal of the plurality of curvilinear sheets may be fast and easy.

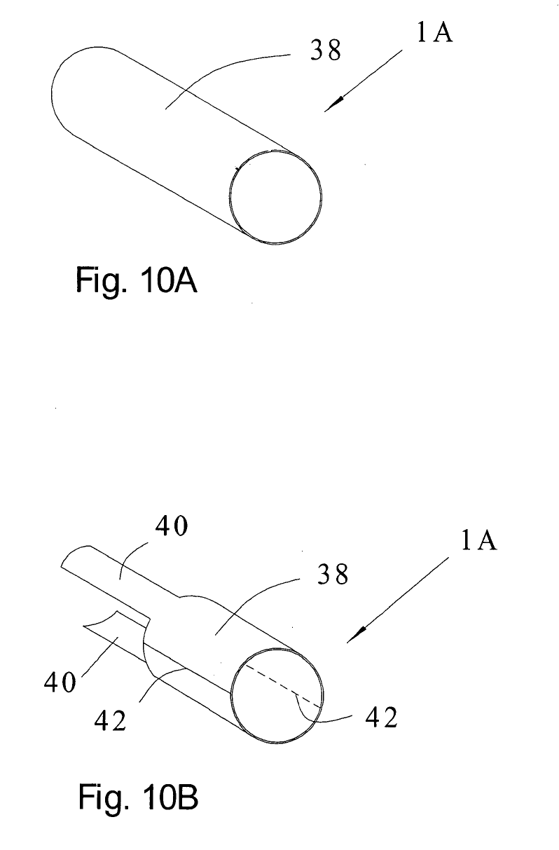

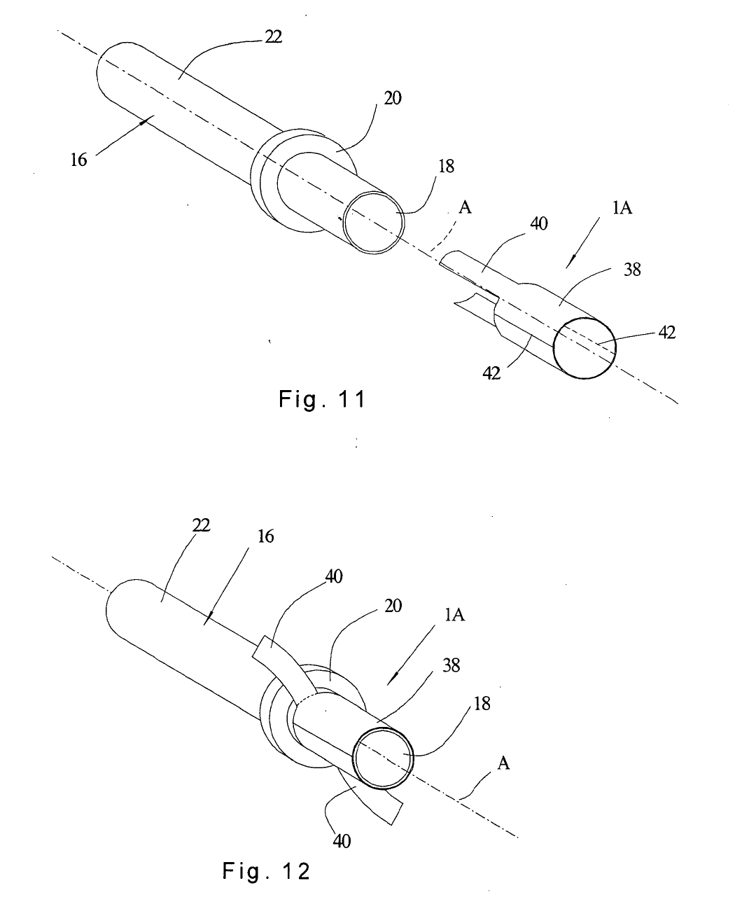

FIGS. 10A-10B are diagrams illustrating a second exemplary embodiment of a conduit protector 1A in accordance with the present invention. As illustrated in FIG. 10A, conduit protector 1A may be formed with a monolithic tubular body 38. Particularly, tubular body 38 may be composed of any suitable, low friction material such as, for example, nylon, polyolefin, or Teflon. Alternatively, as illustrated in FIG. 10B, tubular body 38 may be cut to create one or more handles 40 and one ore more perforation or score lines 42. With these modifications, tubular body 38 may be aligned with adaptor 18 of implant graft 16 such that it is coaxial with the adaptor as illustrated in FIG. 11, and then positioned over adaptor 18 as illustrated in FIG. 12. As further illustrated in FIG. 12, handles 40 of conduit protector 1A may be pushed against front face 26 of cuff 20 such that they are generally perpendicular to the main axis A of tubular body 38.

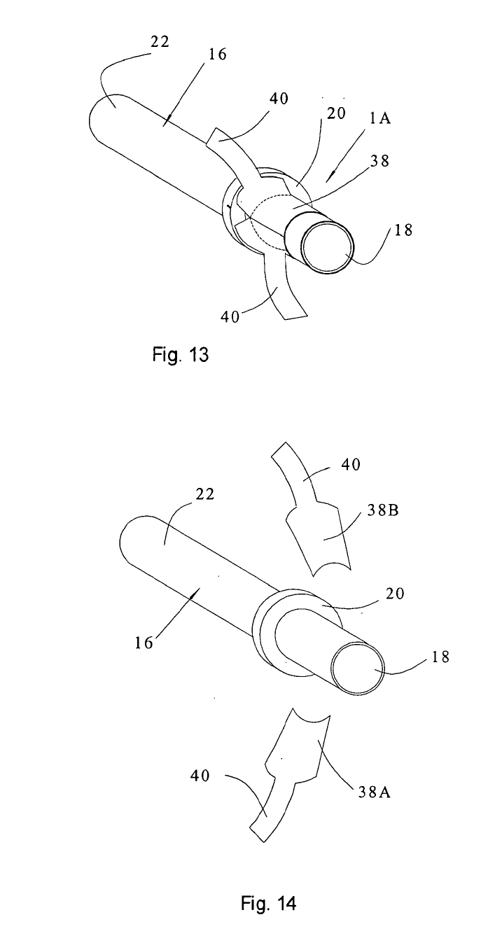

Once conduit protector 1A is in the proper position illustrated in FIG. 12, implant graft 16 may be inserted into a heart wall or any other type of body cavity using a procedure similar to that previously described. Once inserted, conduit protector 1A may be removed by pulling on handles 40 to create a tear or separation along the score lines or perforations 42 as illustrated in FIG. 13. This pulling action may be continued until tubular body 38 of conduit protector 1A is separated into two separate pieces 38A and 38B as illustrated in FIG. 14.

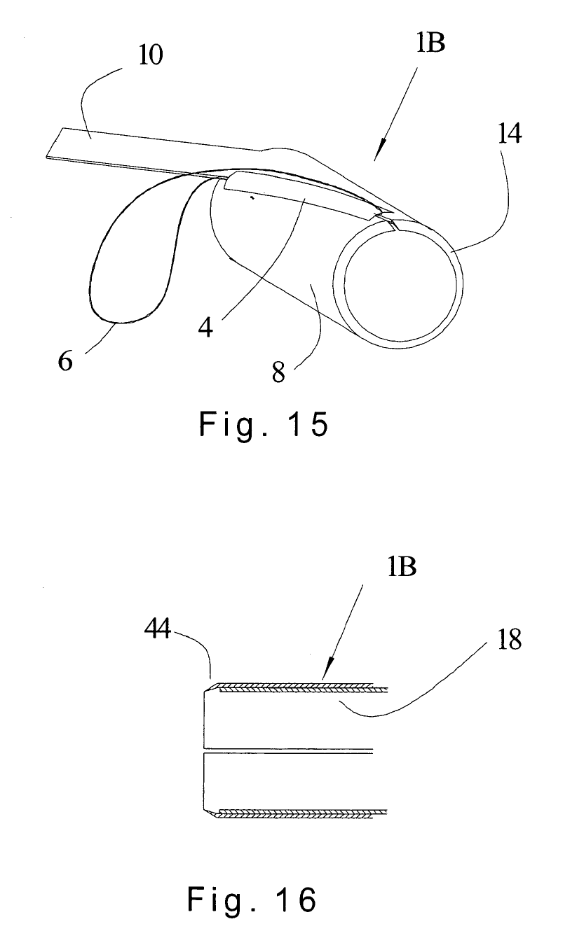

FIG. 15 is a perspective view of a third alternative embodiment of a conduit protector in accordance with the present invention. In particular, conduit protector 1B is similar to conduit protector 1 previously described in reference to FIGS. 1-9, but further includes a tapered distal end 44. As illustrated in FIG. 16, tapered distal end 44 of conduit protector 1B decreases the diameter of the distal tip of the conduit protector such that the inside diameter of the conduit protector may be similar in size to the outside diameter of the hole cut into the wall of the heart or other organ as previously described.

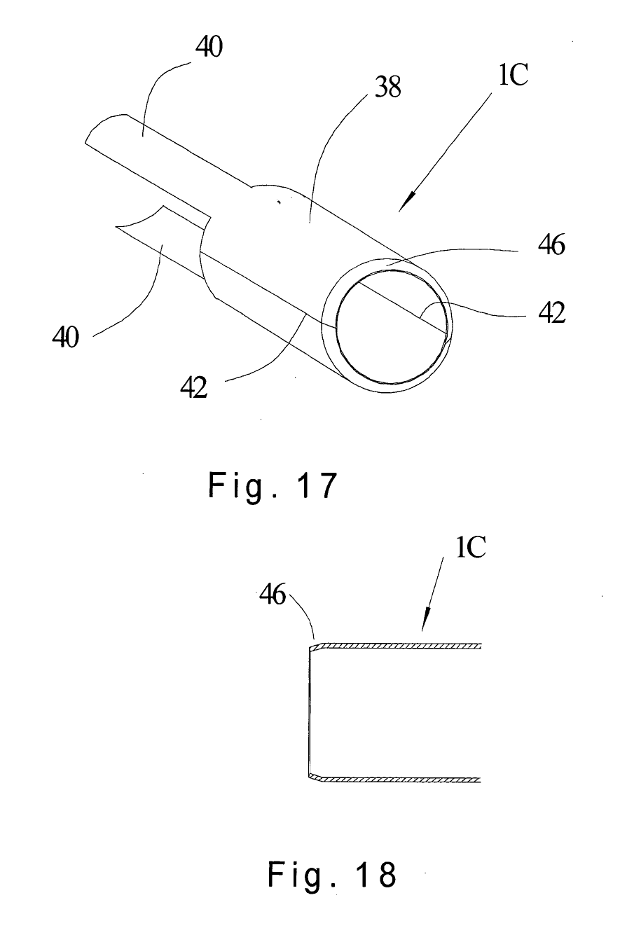

FIG. 17 is a perspective view of a fourth alternative embodiment of a conduit protector in accordance with the present invention. In particular, conduit protector 1C is similar to conduit protector 1A previously described in reference to FIGS. 10-14, but further includes a tapered distal end 46. Similar to tapered distal end 44 of conduit protector 1B, tapered distal end 46 of conduit protector 1C decreases the diameter of the distal tip of the conduit protector as illustrated in FIG. 18. As will be appreciated by those skilled in the art, this tapering of the distal portion of the conduit protectors may provide numerous examples including, but not limited to, reducing both friction and drag during the insertion process.

Although the description above focused on several exemplary embodiments of a conduit protector in accordance with the present invention, those skilled in the art will appreciate that numerous other embodiments are also contemplated and within the intended scope of the present invention. Additionally, reference was made throughout the specification to a conduit protector designed for use in gaining access through the wall of a heart. However, those skilled in the art will appreciate that the protector may be used for numerous other applications where insertion of an implant or other device into other body organs is desired. Furthermore, numerous different materials may be employed to create generally thin walled cylindrical shaped members from sheet stock. Similarly, numerous different connecting and releasing means may be used to create a cylindrical shaped member that may be transformed into a sheet for removal.

Although the present invention has been described with reference to preferred embodiments, workers skilled in the art will recognize that changes may be made in form and detail without departing from the spirit and scope of the invention.

What is claimed:

1. A conduit protector for temporarily minimizing the surface friction on an outside surface of an implant comprising:
a curvilinear sheet, the curvilinear sheet formed into a generally cylindrical shape having a first axial edge, a second axial edge, and an inside surface having an inner diameter approximately equal to an outer diameter of the outside surface of the implant;
a connecting member coupling the first axial edge of the curvilinear sheet to the second axial edge of the curvilinear sheet; and
a release member structured for separating the first axial edge of the curvilinear sheet from the second axial edge of the curvilinear sheet.

2. The conduit protector of claim 1, wherein the ratio between a thickness of the curvilinear sheet and the inner diameter of the generally cylindrical shape is less than about 1:20.

3. The conduit protector of claim 1, wherein the connecting member is an adhesive backed film.

4. The conduit protector of claim 3, wherein the adhesive backed film is formed from a low friction polymer.

5. The conduit protector of claim 1, wherein the release member comprises a thread.

6. The conduit protector of claim 5, wherein the thread is in the shape of a loop.

7. The conduit protector of claim 1, further comprising a handle member extending from the curvilinear sheet.

8. The conduit protector of claim 1, wherein a portion of the release member is disposed between the connecting member and the curvilinear sheet.

9. The conduit protector of claim 1, wherein the curvilinear sheet has a thickness in a range between about 0.001 inches and about 0.020 inches.

10. A conduit protector comprising:
a body portion having a leading edge and a trailing edge;
a handle member extending from the trailing edge of the body portion;
a connecting member coupling the leading edge of the body portion to the trailing edge of the body portion to create a generally cylindrical sheath; and
a release member disposed between the connecting member and the body portion and structured for separating the leading edge of the body portion from the trailing edge of the body portion.

11. The conduit protector of claim 10, wherein the body portion is formed from a low friction polymer material.

12. The conduit protector of claim 10, wherein the body portion has a thickness in a range between about 0.001 inches and about 0.020 inches.

13. The conduit protector of claim 10, wherein the connecting member is a strip of adhesive tape.

14. The conduit protector of claim 13, wherein the adhesive tape is formed from a polymer material having an adhesive on one side.

15. The conduit protector of claim 10, wherein the release member is formed as a loop.

16. The conduit protector of claim 15, wherein the release member comprises one or more segments that are coupled together to form the loop.

17. A conduit protector for minimizing the surface friction on an exterior surface of an implant comprising:
a hollow cylinder including a proximal end, a distal end, an inside surface, and an outside surface;
a pair of score lines extending between the proximal end and the distal end of the cylinder and separating the cylinder into a first cylinder section and a second cylinder section;
a first handle member extending from the first cylinder section; and
a second handle member extending from the second cylinder section.

18. The conduit protector of claim 17, wherein the distal end of the cylinder is tapered.

19. The conduit protector of claim 17, wherein the cylinder is a monolithic cylinder.

20. The conduit protector of claim 17, wherein the pair of score lines extending between the proximal end and the distal end of the cylinder are perforated.
